(19) United States
(12) Patent Application Publication
(10) Pub. No.: US 2010/0249742 A1
(43) Pub. Date: Sep. 30, 2010

(54) COMPARTMENTALIZED INDIVIDUAL PACKAGE FOR ABSORBENT ARTICLES

(76) Inventors: Kimberly Nichole McConnell, Morrow, OH (US); David Andrew Dalton, Mason, OH (US); Virginia Anne Lenahan, Cincinnati, OH (US); Jeanne Marie Hughes, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/414,890

(22) Filed: Mar. 31, 2009

(51) Int. Cl. A61F 13/551; A61B 19/00

(52) U.S. Cl. 604/385.02; 206/440

(57) ABSTRACT

An individually packaged absorbent article including an absorbent article and a package. The package can also have a dispensing compartment substantially enclosing the absorbent article prior to use that can have an opening provided to access the absorbent article. The package can also have a disposal compartment separate from the dispensing compartment. The disposal compartment can have a cavity adapted to receive at least a portion of an absorbent article after use and an opening provided to access the cavity that is separate from the dispensing compartment opening.

### FIELD OF THE INVENTION

This invention relates to individual packages for absorbent articles, and more particularly, to compartmentalized individual packages for absorbent articles.

### BACKGROUND OF THE INVENTION

Absorbent articles, such as absorbent articles for feminine hygiene, are generally used to absorb body exudates, such as menstrual or other body fluids. Certain types of absorbent articles, such as, for example, sanitary napkins, tampons, incontinence pads, and similar articles can be individually packaged. These individual packages can protect the article and can facilitate hygiene, ease of use and identification, ease of carrying, and ease of disposal.

Currently available individual packages can have one or more drawbacks. For example, users of an absorbent article can have a need to dispose of all or a portion of the article after use. While some portions of an absorbent article may be flushable, much of the used article and related material may need to be disposed in a trash bin. Typically, a user can dispose the article or related material directly into the bin, can wrap the used article in toilet tissue for disposal in the bin, or can attempt to use the package of a new article, after the new article is removed, to contain the used article for disposal in the bin. In the event that a trash bin is not convenient to a user, the user may have to wrap and/or carry the used article or material for delivery to a trash bin. Furthermore, when a user prefers to use a new article's package for disposal, the user typically must remove the new article before the used article can be disposed in the package, which can require a user to handle both the new and used article simultaneously during an article change.

As such, it would be desirable to provide an individual package for an absorbent article that can provide for improved disposal of a used article.

### SUMMARY OF THE INVENTION

An individually packaged absorbent article including an absorbent article and a package is provided. The package can have a front face, a back face, a top, a bottom, a first side, a second side, a longitudinal axis, and a transverse axis. The package can also have a dispensing compartment substantially enclosing the absorbent article prior to use and having an opening provided to access the absorbent article, and a disposal compartment separate from the dispensing compartment, the disposal compartment having a cavity adapted to receive at least a portion of an absorbent article after use and an opening provided to access the cavity that is separate from the dispensing compartment opening.

Also provided is an individually packaged absorbent article including an absorbent article and a package. The package can have a front face, a back face, a top, a bottom, a first side, a second side, a longitudinal axis, and a transverse axis. The package can also have a dispensing compartment substantially enclosing the absorbent article prior to use. The dispensing compartment can have a first layer defining an exterior of the package, and a second layer disposed opposite the first layer, the first layer and the second layer being provided to substantially enclose the absorbent article between the first layer and the second layer. The package can also have a disposal compartment separate from the dispensing compartment, the second layer of the dispensing compartment being adapted to provide a portion of the disposal compartment, the disposal compartment further including a third layer, the third layer defining an exterior of the package, the second layer and the third layer providing a cavity adapted to receive at least a portion of an absorbent article between the second layer and the third layer after use.

Further provided is a method of changing an absorbent article. The method includes providing a package having a front face, a back face, a top, a bottom, a first side, a second side, a longitudinal axis, and a transverse axis. The package includes a dispensing compartment substantially enclosing the absorbent article and having an opening provided to access the absorbent article, and a disposal compartment separate from the dispensing component, the disposal compartment having a cavity being adapted to receive at least a portion of the absorbent article after use and an opening provided to access the cavity that is separate from the dispensing compartment opening. The method further includes opening the disposal compartment and disposing at a portion of an absorbent article into the cavity; and opening the dispensing compartment and removing the absorbent article.

### BRIEF DESCRIPTION OF THE DRAWINGS

FIG. 1 is a plan view of an individual package.

FIG. 2 is a plan view of the back of an individual package.

FIG. 3 is a plan view of the front of an individual package.

FIG. 4 is a cross-sectional view of an individual package.

FIG. 5 is a cross-sectional view of an individual package.

### DETAILED DESCRIPTION OF THE INVENTION

The present invention relates to individual packages for absorbent articles, such as, e.g., absorbent articles for feminine hygiene. The individual package can be compartmentalized. In certain embodiments, the individual package can include a disposal compartment for disposal of a used absorbent article along with a separate dispensing compartment that can enclose an absorbent article prior to use. Such package configurations can allow for hygienic storage of a new absorbent article in one compartment and disposal of used material, such as, e.g., a used tampon applicator, used absorbent article, and/or related material, in a separate compartment. In addition, the package can be configured to received a used absorbent article or related material prior to release of the new article, which can provide for an easier and/or cleaner absorbent article changing experience. For example, in certain embodiments, the individual package can include a disposal compartment that can be opened separately and prior to opening and release of a new absorbent article form the dispensing compartment and can be configured to facilitate disposal of used material, such as, e.g., a disposal compartment including an absorbent material for retaining residual body fluids and/or for reducing soiling of a user's hands, a larger opening for easy disposal of the used material, and/or a mechanism, such as, e.g., a sealing tab, for closing the used material inside the disposal compartment.

As used herein, the term “absorbent article” refers to devices which absorb and contain body exudates, and more specifically, refers to devices that are placed against or in proximity to the body of the wearer to absorb and contain various exudates discharged from the body. Absorbent articles include, for example, sanitary napkins, incontinence articles, interlabial pads, tampons, and panty liners.

The term “disposable” is used herein to describe articles that are intended to be discarded after a single use. That is, they are not intended to be laundered or otherwise restored or reused.

As used herein, the term “absorbent” refers to materials that can hold, retain, and/or contain body fluids.

As used herein, the term “feminine hygiene articles” refers to articles that typically can be intended for feminine use, such as, e.g., absorbent articles, such as, e.g. sanitary napkins, liners, tampons, interlabial articles, incontinence articles; and pessaries.

As used herein, the term “tampon” refers to any type of absorbent structure such as, e.g., an absorbent mass, that can be inserted into the vaginal canal or other body cavity for the purpose of absorbing fluid, aiding in wound healing, and/or for delivering materials, such as moisture or active materials such as medicaments. The term “tampon” can also include the combination of an absorbent structure with any type of applicator that can be associated with the absorbent structure to facilitate insertion of a tampon into the vaginal canal or other body cavity. A tampon can include any known tampon configuration such as, for example, digital tampons, tampons with traditional plunger type applicators, and/or tampons with compact applicators, such as, e.g., tampons described in U.S. Pat. Nos. 4,726,805; 4,846,802; 4,960,417; 5,087,239; 5,279,541; 6,258,075; 6,478,763; or any other tampon.

FIG. 1 shows one embodiment of an individual package 10. The package 10 has a front face 11, a back face 12, a top 13, a bottom 14, a length (l), a width (w), a longitudinal axis (L), and a transverse axis (T). As shown in FIG. 1, package 10 can also have a first side 15 and a second side 16 that can include side seals 17. In addition, in certain embodiments, package 10 can include a flap 21 that can include a sealing tab 22.

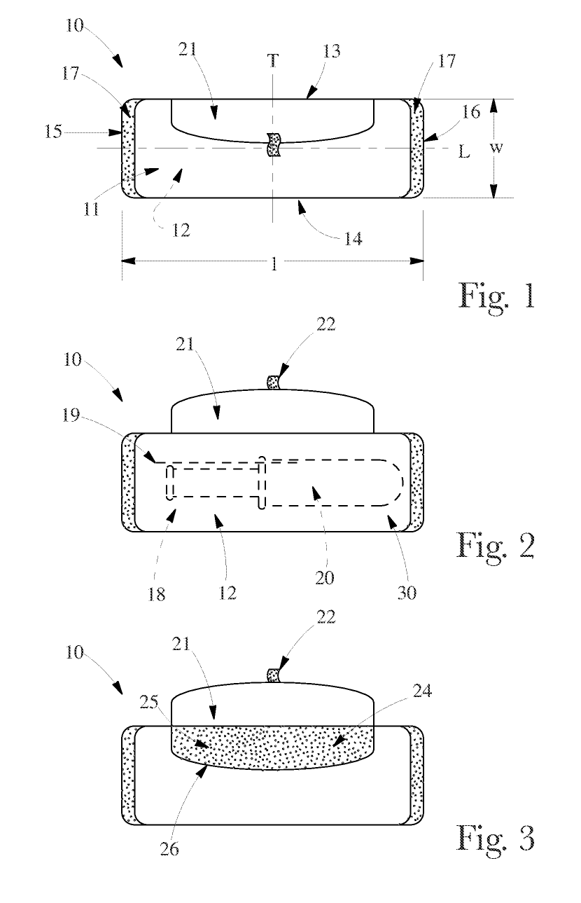

FIG. 2 shows one embodiment of an individual package 10. As shown in FIG. 2, package 10 can have a dispensing compartment 18 that can substantially enclose an absorbent article 20. In certain embodiments, package 10 can include a line of weakness 19 that can be provided to allow access to absorbent article 20 enclosed within dispensing compartment 18. As shown in FIG. 2, the line of weakness can be provided on a first wall 30 of material forming a portion of the dispensing compartment 18, such as, e.g., a first wall 30 that can also form the back face 12 of the package 10.

FIG. 3 shows one embodiment of an individual package 10. As shown in FIG. 3, package 10 can have a disposal compartment 24 that can be adapted to contain a used absorbent article or a portion thereof. Disposal compartment 24 can be accessed by an opening 25. In certain embodiments, opening 25 can be formed by breaking one or more perforations 26. Alternatively, in certain embodiments, opening 25 can be an open space, such as, e.g., a gap, a slit, a slot, a cavity, or any other suitable opening. In certain embodiments, package 10 can include a flap 21 that can be provided to cover opening 25 and/or to seal disposal compartment 24 after used material is placed therein. In addition, in certain embodiments, flap 21 can include a sealing tab 22.

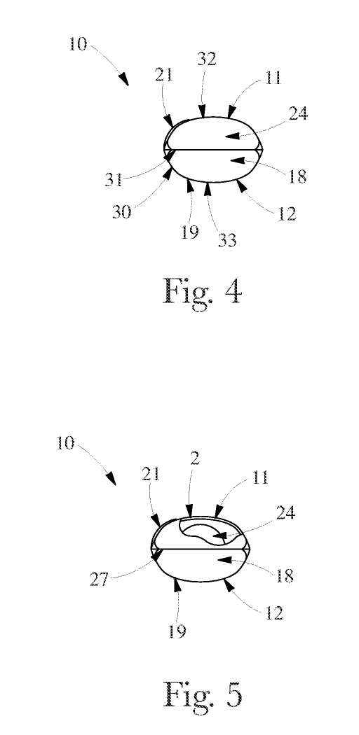

FIG. 4 shows a cross-sectional view of an individual package 10. The package 10 can have a front face 11, a back face 12, a dispensing compartment 18, and a disposal compartment 24. In certain embodiments, package 10 can include a flap 21 for accessing a compartment of the package, such as, e.g., for accessing disposal compartment 24. In addition, or alternatively, package 10 can include a line of weakness 19 that can be provided to access a compartment of the package, such as, e.g., dispensing compartment 18. As shown in FIG. 4, dispensing compartment 18 can have a first layer 30 that can define an exterior of the package 33. In addition, dispensing compartment 18 can have a second layer 31 disposed opposite first layer 30 configured to substantially contain an absorbent article between first layer 30 and second layer 31. As shown in FIG. 4, in certain embodiments, second layer 31 can be adapted to also provide a portion of disposal compartment 24. Disposal compartment 24 can further include a third layer 32 that can define an exterior of the package 34. In certain embodiments, second layer 31 and third layer 32 can be adapted to receive at least a portion of an absorbent article 20 between second layer 31 and third layer 32, such as, e.g., after use of the article.

FIG. 5 shows a cross-sectional view of an individual package 10. The package 10 can have a front face 11, a back face 12, a dispensing compartment 18, and a disposal compartment 24. In certain embodiments, package 10 can include a flap 21 for accessing a compartment of the package, such as, e.g., for accessing disposal compartment 24. In addition, or alternatively, package 10 can include a line of weakness 19 that can be provided to access a compartment of the package, such as, e.g., dispensing compartment 18. As shown in FIG. 5, in certain embodiments, package 10 can be formed of a first material 26. In addition, as shown in FIG. 5, disposal compartment 24 can include one or more additional materials 27, that can be, for example, one or more absorbent materials, such as, e.g., one or more nonwoven materials.

The dispensing compartment and the disposal compartment can be generally accessed by separate openings. For example, the dispensing compartment will typically contain the new absorbent article and as such, can enclose the new article to promote hygiene and protect the article prior to use. In certain embodiments, the dispensing compartment can be substantially sealed around the absorbent article until the article is released by a user, such as, e.g., by breaking a line of weakness, releasing a seal and/or tab, or other suitable means for accessing the article contained within the dispensing compartment. The disposal compartment, on the other hand, can be configured to provide a cavity that can receive a used absorbent article or related material. In certain embodiments, the disposal compartment can be accessed by an opening suitable for easily disposing the used article. In addition, or alternatively, the disposal compartment can be empty and need not be substantially sealed prior to disposal of the used article therein, which can, for example, provide easy disposal into the disposal compartment. In certain embodiments, the disposal compartment can include a tab, such as a resealable tab, for holding the disposal compartment opening closed prior to use and/or after the used article and/or related material is disposed therein.

In certain embodiments, a user can remove a used absorbent article and dispose it within the disposal compartment, release a new absorbent article from the dispensing compartment, apply the new absorbent article, and dispose of any residual material, such as, e.g., a used tampon applicator, release strips, or other related material, within the disposal compartment. Alternatively, a user can release the new absorbent article from the dispensing compartment, apply the new absorbent article, and dispose of any residual material within the disposal compartment. In addition, in certain embodiments, the user can seal the disposal compartment to contain the used absorbent article and/or related material. In certain embodiments, the disposal compartment can include a resealable tab, such that the user can open the disposal compartment, seal the disposal compartment after placing a used absorbent article therein, reopen the disposal compartment to include any additional material, and reseal the compartment for delivery to the trash bin. For example, when the package is a tampon package for a single tampon and applicator, the user can remove her used tampon if necessary, flush the used tampon or dispose of it within the disposal compartment, release the new tampon and applicator from the dispensing compartment, insert the tampon using the applicator, place the used applicator into the disposal compartment along with the used tampon, seal the disposal compartment, and dispose of the package in a trash bin.

The dispensing compartment and the disposal compartment can be provided in any suitable configuration and in any suitable manner. For example, in certain embodiments, the dispensing compartment and the disposal compartment can be provided in a back to front relationship, with the dispensing compartment at the back of the wrapper and the disposal compartment at the front of the wrapper, or vice versa, or in a side to side relationship. In certain embodiments, the disposal compartment and/or the dispensing compartment can be defined by a material that can also form all or a portion of an exterior wall of the individual package and/or by a material that can form all or a portion of an interior wall of the individual package. In addition, in certain embodiments, the disposal compartment and the dispensing compartment can share a wall of the package, such as, e.g., an interior wall. For example, in certain embodiments, the dispensing compartment can be formed by material that defines the back of the individual package, and by material that forms an interior wall of the package that separates the dispensing compartment from the disposal compartment, and the disposal compartment can be formed by material corresponding to the interior wall and by material defining the front of the individual package.

The separate openings for each compartment can be provided in any suitable location. For example, in certain embodiments, access to the disposal compartment and access to the dispensing compartment can be provided on different surfaces of the package, such as, e.g., on opposite faces of the package. The disposal compartment opening and the dispensing compartment opening can be any suitable configuration, such as, e.g., in the same configuration as each other or as different from each other. For example, in certain embodiments, the disposal compartment opening and the dispensing compartment opening can both include one or more lines of weakness. Alternatively, the disposal compartment opening may be an open space and the dispensing compartment opening can include one or more lines of weakness. In addition, in certain embodiments, the disposal compartment opening can include one or more sealing features that can function to seal the disposal opening after a used article or related material is disposed therein.

The openings can be any suitable length. For example, the dispensing opening can be a length suitable for removal of the new absorbent article. In addition, or alternatively, the disposal compartment opening can be any length suitable for disposing a used article or related material therein, such as, e.g., greater than about 50% of the length of the package, greater than about 75%, greater than about 95%, substantially the entire length of the package, or any other suitable length for disposing a used article or related material.

In certain embodiments, the dispensing compartment and the disposal compartment can be made from the same material. In addition, or alternatively, the dispensing compartment and/or the disposal compartment can include one or more additional or different materials. For example, in certain embodiments, the disposal compartment can include additional features, such as, e.g., one or more absorbent materials provided on the interior of the disposal compartment that can, for example, act to absorb and/or retain any body exudates from the used absorbent article. In addition, or alternatively, the disposal compartment and/or the dispensing compartment can include odor control features.

The package can be formed in any suitable configuration. Suitable configurations can include, for example, a tampon package, a sanitary napkin package, an incontinence pad package, a liner package, or any other suitable configurations. In certain embodiments, for example, the package can be an individual package for a single tampon, and the package can be formed by folding the package material along a bottom fold line substantially parallel to the transverse or longitudinal axis to form the bottom of the package, and by folding the package material along a top fold line substantially parallel to the bottom fold line. In addition, the sides of the package can then be sealed.

The package can be comprised of any material suitable for packaging individual absorbent articles. Suitable materials include, for example, flexible materials, such as, e.g., flexible polymeric films, such as, e.g. polymeric films made of polyethylene, polypropylene, polyester, cellophane, polyamide, polyvinyl chloride, ethylene-vinyl acetate copolymer, and/or other suitable films. In certain embodiments, the package material can be heat-shrinkable films, stretch films or pre-stretched elastic material, such as, e.g., package material that comprises polyolefins such as polyethylene and polypropylene, or polyvinyl chloride. Other suitable materials include, e.g. polystyrene, polyethylene-terephtalate (PET), and metallic foils, such as aluminium foil. Suitable materials can also include, e.g. non-occlusive or porous materials such as nonwovens, wovens, scrims, meshes and papers. Such non-occlusive materials can be made occlusive such as by lamination with or by coating with occlusive material. In the case of cellulosic papers, examples include lamination with a polymeric film such as a polyolefinic composition or coating or impregnation of the paper with wax. In certain embodiments, package materials can be coated with various chemical compounds to improve their barrier properties or the ability for sealing. In addition, or alternatively, the package material can have a low flexural modulus for providing a low noise package during transport as well as during handling and opening of the package.

The package can be constructed in any suitable manner, such as, e.g., constructed of one connected piece of package material or constructed from multiple pieces of material sufficiently joined together such that it substantially acts as one connected piece of package material. In certain embodiments, the package can be formed by closing the package material via heat-sealing onto itself after wrapping the absorbent article. In addition, or alternatively, the package can be glued, embossed, crimped, sewed, stitched, entangled, mechanically interlocked, cold pressure welded, ultrasonic bonded, and/or otherwise bonded or sealed.

In certain embodiments, the package can include a line of weakness that can function, for example, to allow access to a compartment. A line of weakness can be formed in any suitable manner, such as, e.g., mechanically and/or thermally, such as, for example, by using a laser and/or chemically. A line of weakness can include a plurality of weakness points arranged in a row. The row can be straight, bent, angled, curved, and/or can change direction. In certain embodiments, one or more of the individual weakness points can overlap to form the line of weakness. In addition, or alternatively, one or more of the individual weakness points can be spaced apart from one another to form the line of weakness. The one or more lines of weakness can be formed by perforations or by depressions.

In certain embodiments, the package can include a device for sealing a portion of the package over the used article once it is placed into the package for transportation and/or disposal. Any suitable device for sealing the package can be used, such as, e.g., adhesive, glues, tapes, thermal bonds, and/or mechanical fasteners. In certain embodiments, the sealing device can include a portion of the surface of the package that can be covered with adhesive, cohesive, or other joining means. In addition, or alternatively, the device can include a sealing tab that can have, for example, a pressure sensitive adhesive or other suitable closure means disposed on one or more surfaces of the sealing tab.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An individually packaged absorbent article comprising:
an absorbent article;
a package having a front face, a back face, a top, a bottom, a first side, a second side,
a longitudinal axis, and a transverse axis,
a dispensing compartment substantially enclosing the absorbent article prior to use and having an opening provided to access the absorbent article,
a disposal compartment separate from the dispensing compartment, the disposal compartment having a cavity adapted to receive at least a portion of an absorbent article after use and an opening provided to access the cavity that is separate from the dispensing compartment opening.

2. The individually packaged absorbent article of claim 1, wherein the dispensing compartment opening is provided on a first face of the package and the disposal compartment opening is provided on a second face of the package.

3. The individually packaged absorbent article of claim 1, wherein the dispensing compartment opening includes a line of weakness.

4. The individually packaged absorbent article of claim 1, wherein the disposal compartment opening is unsealed.

5. The individually packaged absorbent article of claim 1, wherein the disposal compartment opening extends across greater than at least about 50% of the length of the package.

6. The individually packaged absorbent article of claim 1, wherein the dispensing compartment opening is provided on the back face of the package and the disposal compartment opening is provided on the front face of the package.

7. The individually packaged absorbent article of claim 1, wherein the absorbent article is a tampon, a sanitary napkin, an interlabial pad, or an incontinence pad.

8. The individually packaged absorbent article of claim 1, wherein the package is formed from a plastic film and wherein the disposal compartment further includes one or more absorbent materials.

9. The package of claim 1, wherein the disposal compartment is resealable.

10. An individually packaged absorbent article comprising:
an absorbent article;
a package having a front face, a back face, a top, a bottom, a first side, a second side,
a longitudinal axis, and a transverse axis,
a dispensing compartment substantially enclosing the absorbent article prior to use, the dispensing compartment having a first layer defining an exterior of the package, and a second layer disposed opposite the first layer, the first layer and the second layer being provided to substantially enclose the absorbent article between the first layer and the second layer;
the package having a disposal compartment separate from the dispensing compartment, the second layer of the dispensing compartment being adapted to provide a portion of the disposal compartment, the disposal compartment further including a third layer, the third layer defining an exterior of the package, the second layer and the third layer providing a cavity adapted to receive at least a portion of an absorbent article between the second layer and the third layer after use.

11. The individually packaged absorbent article of claim 10, wherein the package includes a line of weakness disposed to provide access to the dispensing compartment.

12. The individually packaged absorbent article of claim 10, wherein the package includes an opening provided to access the disposal compartment, the opening extending across greater than at least about 50% of the length of the package.

13. The individually packaged absorbent article of claim 10, wherein the absorbent article is a tampon, a sanitary napkin, an interlabial pad, or an incontinence pad.

14. The individually packaged absorbent article of claim 10, wherein the first layer is formed from a plastic film and the second layer is formed from one or more absorbent materials.

15. The individually packaged absorbent article of claim 10, wherein the disposal compartment is resealable.

16. A method of changing an absorbent article, the method comprising the steps of:
a) providing a package having a front face, a back face, a top, a bottom, a first side, a second side, a longitudinal axis, and a transverse axis, a dispensing compartment substantially enclosing the absorbent article and having an opening provided to access the absorbent article, a disposal compartment separate from the dispensing component, the disposal compartment having a cavity being adapted to receive at least a portion of the absorbent article after use and an opening provided to access the cavity that is separate from the dispensing compartment opening;
b) opening the disposal compartment and disposing at a portion of an absorbent article into the cavity; and
c) opening the dispensing compartment and removing the absorbent article.

17. The method of claim 16, wherein the dispensing compartment opening is provided on a first face of the package and the disposal compartment opening is provided on a second face of the package.

18. The method of claim 16, wherein the method further includes sealing the disposal compartment to enclose the used absorbent article.

19. The method of claim 16, wherein the package is formed from a plastic film and wherein the disposal compartment further includes one or more absorbent materials.

20. The method of claim 16, wherein the absorbent article is a tampon, a sanitary napkin, an interlabial pad, or an incontinence pad.
